(19) United States
(12) Patent Application Publication
(10) Pub. No.: US 2023/0395189 A1
(43) Pub. Date: Dec. 7, 2023

(54) Genome-Wide Detection of Chromatin interactions in Re-Arranged Genomes

(71) Applicants: Feng Yue, Chicago, IL (US); Xiaotao Wang, Chicago, IL (US)

(72) Inventors: Feng Yue, Chicago, IL (US); Xiaotao Wang, Chicago, IL (US)

(21) Appl. No.: 17/803,371

(22) Filed: Jun. 1, 2022

(51) Int. Cl. G16B 20/10; G16B 40/00; G16B 30/00; C12Q 1/6869

(52) U.S. Cl. CPC G16B 20/10; G16B 40/00; G16B 30/00; C12Q 1/6869

(57) ABSTRACT

The invention relates to a novel computer-implemented computational method that can predict structural variation (SV) induced chromatin interactions, such as inter-chromosomal translocations, large deletions, and inversions, which can be used to identify critical oncogenic regulatory elements for tumorigenesis and potentially reveal therapeutic targets.

### STATEMENT REGARDING FEDERALLY FUNDED RESEARCH AND DEVELOPMENT

This invention was made with government support under grant numbers R35GM124820 and RO1HG009906awarded by the National Institutes of Health. The United States government has certain rights to this invention.

### INCORPORATION OF SEQUENCE LISTING

The present application contains a sequence listing submitted through the EFS-Web and the full text of which is incorporated herein by reference. The ASCII copy generated on Jan. 12, 2023 is named SequenceListing.txt, and the size is 2,751 bytes.

### BACKGROUND OF THE INVENTION

Structural variations (SVs), such as deletions, inversions and translocations, frequently occur in cancer genomes, and have been shown to play a vital role in tumorigenesis (Futreal, P. A. et al., Nat Rev Cancer 4, 177-183 (2004)). Most of the previous studies have focused on how SVs affect protein-coding genes, including formation of the oncogenic fusion genes. However, recent efforts by the ENCODE (Consortium, E. P., Nature 489, 57-74 (2012)) Consortium and the Roadmap Epigenomics Project (Bernstein, B. E. et al., Nat Biotechnol 28, 1045-1048 (2010)) have shown that there are millions of potential distal regulatory elements such as enhancers in the human genome. SVs have been shown to juxtapose enhancers to key cancer genes and contribute to their aberrant elevated expression, which is termed “enhancer hijacking” (Weischenfeldt, J. et al., Nat Genet 49, 65-74 (2017); Spielmann, M., Lupianez, D. G. & Mundlos, S., Nat Rev Genet 19, 453-467 (2018)).

For example, inversion inv(3)(q21q26.2) has been recurrently observed in acute myeloid leukemia (AML), in which the GATA2 enhancer is repositioned near the oncogene EVI1 and ectopically activates EVI1 expression (Groschel, S. et al., Cell 157, 369-381 (2014)). In adenoid cystic carcinoma (ACC), translocations reposition a super-enhancer in proximity to the MYB gene, activating its elevated expression (Drier, Y. et al., Nat Genet 48, 265-272 (2016)). Further, structural variations can induce “neo-TADs” in developmental diseases (Franke, M. et al., Nature 538, 265-269 (2016)) and a subgroup of medulloblastoma (Northcott, P. A. et al., Nature 547, 311-317 (2017)).

Despite its importance, “enhancer hijacking” has only been mainly reported in case studies (Groschel, S. et al., Cell 157, 369-381 (2014); Drier, Y. et al., Nat Genet 48, 265-272 (2016); Yang, M. et al., Blood (2020); Ooi, W. F. et al., Gut 69, 1039-1052 (2020); Martin-Garcia, D. et al., Blood 133, 940-951 (2019); Haller, F. et al., Nat Commun 10, 368 (2019); Zimmerman, M. W. et al., Cancer Discov 8, 320-335 (2018); Ryan, R. J. et al., Cancer Discov 5, 1058-1071 (2015); Northcott, P. A. et al., Nature 511, 428-434 (2014)). To date, no computational tools exist to identify such events from chromatin interaction data, such as Hi-C. CESAM (Weischenfeldt, J. et al., Nat Genet 49, 65-74 (2017)) and PANGEA (He, B. et al., Sci Adv 6, eaba3064 (2020)) can predict enhancer-hijacking events genome-wide, by building a regression model of gene expression with SV profiles from hundreds or thousands of genomes. Such methods, while valuable for large cohorts of samples, cannot be directly applied when only one or a small number of samples are available.

Disclosed herein is a computational framework to identify the chromatin interactions induced by SVs, such as inter-chromosomal translocations, large deletions, and inversions. Specifically this method can
a) automatically resolve complex SVs;
b) reconstruct local Hi-C maps surrounding the breakpoints;
c) normalize copy number variation and allele effects; and
d) predict chromatin loops induced by SVs.

The advantages of this computational framework include:
a) It increases the quality of the input SVs, which the accuracy of local assemblies and reconstructed Hi-C maps rely on;
b) It can reconstruct different Hi-C maps for different alleles when heterozygous SVs are provided;
c) It relies on its machine-learning based method, rather than testing for significant enrichment of Hi-C signals compared to a local or global background (Salameh, T. J. et al., Nat Commun 11, 3428 (2020));
d) Its embedded visualization module is the first stand-alone automatic tool to display rearranged-chromatin structures along with genes and epigenomic tracks for any SVs or complex SVs. With the continuous growth of Hi-C data in cancer, a 3D cancer browser can be built upon the 3D genome browser (Wang, Y. et al., Genome Biol 19, 151, (2018)) to provide a comprehensive toolkit for interactive visualization and data integration of cancer 3D genomics data.

### SUMMARY OF THE INVENTION

The inputs to the algorithm are a Hi-C contact matrix and a list of SV breakpoints, which can be identified from different platforms such as Hi-C, whole-genome sequencing (WGS), and optical mapping (Dixon, J. R. et al., Nat Genet 50, 1388 (2018)). The computational framework will output the following: 1) genome-wide CNV profile; 2) genome-wide CNV segments; 3) a chain of linked SV events (local assembly); 4) corrected Hi-C matrix for the newly assembled regions; 5) chromatin loops in the rearranged regions. 6) Enhancer-hijacking events when H3K27ac data are available.

In accordance with a preferred embodiment of the invention, it has been found that a modified matrix balancing algorithm can remove CNV's distorting effects on Hi-C signals along with other systematic biases including mappability, GC content, and restriction fragment sizes (STEP 1 in FIG. 1A). The algorithm begins with the inference of genome-wide CNV profiles and CNV segments from Hi-C maps, and then applies matrix balancing to regions with different copy numbers separately.

In accordance with a preferred embodiment of the invention, it has been found that a graph-based algorithm can assemble the correct order of each individual SVs in order to resolve the complex SVs formed by a chain of SVs (FIG. 1B and FIGS. 2B-F). To reconstruct the Hi-C matrix for the rearranged genomic regions (STEP 2 in FIG. 1A), submatrices from different parts of the reference map are flipped or rotated according to the types and orientations of the breakpoints (FIG. 2A). While local complex SV structures can be manually reconstructed, the process requires WGS, optical mapping, and Hi-C (Dixon, J. R. et al., Nat Genet 50, 1388 (2018)). This algorithm automatically assembled all these complex SVs solely based on Hi-C data (FIG. 3).

In accordance with a preferred embodiment of the invention, it has been found that a linear-regression model can scale signals from different regions into a similar range (STEP 3 in FIG. 1A and FIGS. 4A-C). That helps remove the bias in the originally reconstructed Hi-C map caused by the allelic effect.

In accordance with a preferred embodiment of the invention, it has been found that the machine-learning based framework Peakachu (STEP 4 in FIG. 1A) (Salameh, T. J. et al., Nat Commun 11, 3428 (2020)) can be used to train models at various resolutions and window sizes, covering both in situ and dilution Hi-C protocols. The products from this framework include both loops in the regions not affected by SVs and chromatin loops induced by SVs (neo-loops). These neo-loops will serve as the basis for the detection of enhancer hijacking events. Chromatin loop predictors are also tested without removing CNV effect or reconstructing local Hi-C maps surrounding SVs (FIG. 5), suggesting the necessity of considering CNVs and SVs in analyzing chromatin interaction data in rearranged genomes.

In accordance with a preferred embodiment of the invention, it has been found that a visualization module can facilitate the integration with other omics data, which provides functions such as plotting triangular contact heatmaps, genes, epigenomic tracks, and loops, using SV assembly coordinates with correct orientations.

### BRIEF DESCRIPTION OF THE DRAWINGS

FIG. 1A and 1B illustrate the overall design of the computational framework.

FIGS. 2A, 2B, 2C, 2D, 2E, and 2F illustrate complex SV detection based on Hi-C maps.

FIG. 3 shows example of complex SVs assembled by this computational framework using Hi-C data.

FIGS. 4A, 4B, and 4C illustrate cancer-specific allele normalization and neo-TAD identification

FIG. 5 shows comparison of loops predicted by FitHiC2 and this computational framework in SV regions.

FIGS. 6A, 6B, 6C, 6D, 6F, and 6G show the evaluation of the CNV segmentation and CNV normalization module.

FIG. 7 evaluates the performance of CNV normalization of Hi-C data by simulation.

FIGS. 8A, 8B, 8C, 8D, and 8E illustrate cancer-type specificity of the neo-loop-involved genes.

FIG. 9 shows examples of neo-loop-involved genes.

FIGS. 10A and 10B illustrate analysis of the expression of the genes in neo-loops.

FIG. 11 illustrates genes with hijacked enhancers are up-regulated in cancer.

FIGS. 12A, 12B, 12C, and 12D illustrate deletion of hijacked enhancers reduced oncogene expression in prostate adenocarcinoma.

FIG. 13 illustrates application of this computational framework to developmental diseases with genomic rearrangements.

FIGS. 14A, 14B, and 14C illustrate reconstruction of complex SVs in 50 cancer samples.

FIGS. 15A, 15B, 15C, and 15D illustrate detection of neo-loops in 50 cancer cell lines or patient samples.

### DETAILED DESCRIPTION OF THE INVENTION

Disclosed herein is a novel computational framework aiming at the identification of chromatin interactions from Hi-C map in cancer and other diseases with genome rearrangements. To date, there is no computational method to identify enhancer hijacking events directly from genome-wide chromatin interaction data such as Hi-C (Lieberman-Aiden, E. et al., Science 326, 289-293 (2009)). Given Hi-C contact matrices and SV breakpoints in the genome, this method can automatically resolve complex SVs, reconstruct local Hi-C maps surrounding the breakpoints, normalize copy number variation and allele effects, and predict chromatin loops induced by SVs.

The method generally includes the following steps:

a) Copy Number Inference from Hi-C Map

The method is embedded with a copy number inference module to detect copy number variations (CNVs) directly from Hi-C map at a given resolution. The whole process takes two steps:

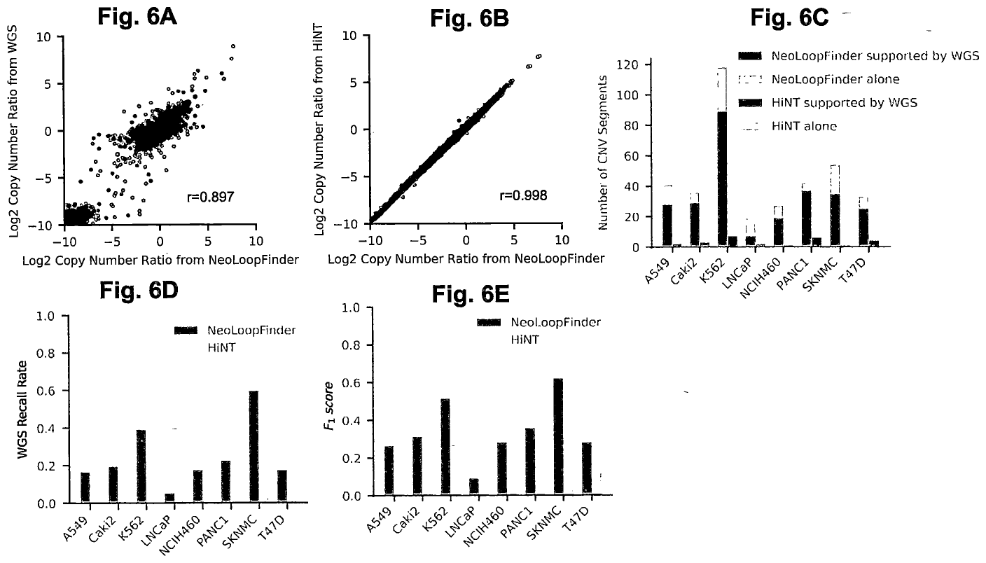

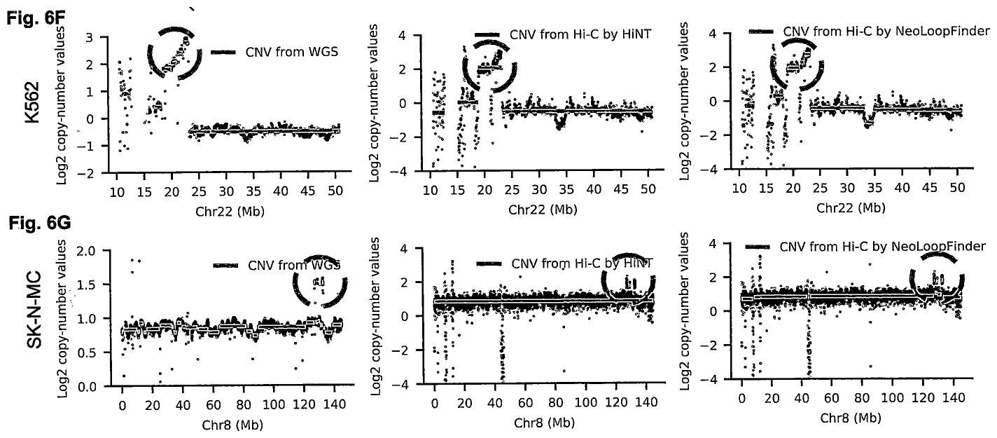

In the first step, the generalized additive model (GAM) proposed by HiNT-CNV20 to model the non-linear relationship between the one-dimensional coverage profile and different biases commonly observed in a Hi-C experiment, including GC content, mappability and the density of restriction sites is implemented. The residuals from the GAM model are used as an initial estimate of the copy number ratio for each bin. It has been confirmed that the results for this step are nearly identical to the output from HiNT-CNV and well correlated with the profile calculated from whole-genome sequencing (FIGS. 6A-B).

In the second step, a Hidden Markov Model (HMM) based segmentation algorithm is used to determine the boundaries of CNV segments from the initial CNV profile. The original segmentation algorithm used by HiNT-CNV (BIC-Seq) tends to report large blocks of amplified/depleted regions (FIGS. 6F-G), which caused inaccurate CNV normalization of Hi-C matrix. In this HMM model, the initial CNV profile outputted from the first step is taken as the observed sequence, while the real copy number of a fragment is treated as the hidden state. For each chromosome, the method first determines the best number of possible states N (the maximum number is set to 15) with the Gaussian Mixture Model and the BIC criterion. Then an HMM model is built using the pomegranate (https://github.com/jmschrei/pomegranate) Python package, with each state approximated by a 2-component Gaussian mixture. Before training, the method detects and removes the outliers within the raw CNV profile with the Hampel filter and split it by 0 s. The Baum-Welch algorithm is used to estimate the parameters of transition and emission and the Viterbi algorithm is used to predict the state (copy number) of each bin. Finally, the consecutive bins assigned with the same state are merged together to form a CNV segment. By default, the copy number of a segment is defined as the average copy number ratios. If ploidy is known, the absolute copy number will be further calculated by multiplying the copy number ratio by the ploidy and rounding to the nearest integer.

### b) Separate Matrix Balancing and CNV Effect Correction

In a traditional matrix balancing or ICE algorithm, the normalized Hi-C map Mᵢⱼ* is modeled identically for the whole genome as CᵢMᵢⱼCⱼ, where Cᵢ is the bias vector corresponding to each bin, which can be solved via an iterative correction approach. This normalization algorithm can remove most biases in Hi-C, including mappability, GC content and restriction fragment sizes (Imakaev, M. et al., Nat Methods 9, 999-1003 (2012)). Its efficiency and easy-to-implementation features have made it a standard and necessary step in most Hi-C processing software. However, ICE-normalized signal intensity in amplified regions can be much lower than in regions with normal copies. Applying ICE or matrix balancing to cancer Hi-C can yield biased values due to amplification/depletion of the genome.

The method proposes a modified ICE procedure by applying ICE to regions with different copy numbers separately:
STEP 1. Initialize an intermediate matrix Mᵢⱼ′=Mᵢⱼ and the bias vector Cᵢ filled with ones.
STEP 2. Calculate the marginal sums of Mᵢⱼ′: ΣᵢMᵢⱼ′;
STEP 3. Extract all bins with copy number k, and calculate the bias vector as below:

$${C}_{i} = \frac{{C}_{i} * {scale}^{k}}{{\sum }_{j} {M}_{ij}^{′}}$$

where scale^{k} represents the average of non-zero marginal sums of bins with copy number equaling to k.
STEP 4. Iterate all possible copy numbers and repeat STEP 3.
STEP 5. Update Mᵢⱼ′ using following equation:

Mᵢⱼ′=CᵢMᵢⱼCⱼ
STEP 6. Repeat STEP 1-5 until the average of variance of marginal sums for bins with identical copy numbers is less than 1e-5.
STEP 7. Scale Cᵢ by Cᵢ=Cᵢ/\sqrt{scale^{k}}, where CNV(i)=k
STEP 8. The normalized Hi-C map can be calculated by multiplying the final bias vector Mᵢⱼ*=CᵢMᵢⱼCⱼ.

The implementation is built upon the cool format, and the bias vector returned by the modified ICE above is stored under the “sweight” column in the input cool file.

### c) Simulation of CNV Effects on a Normal Hi-C Map

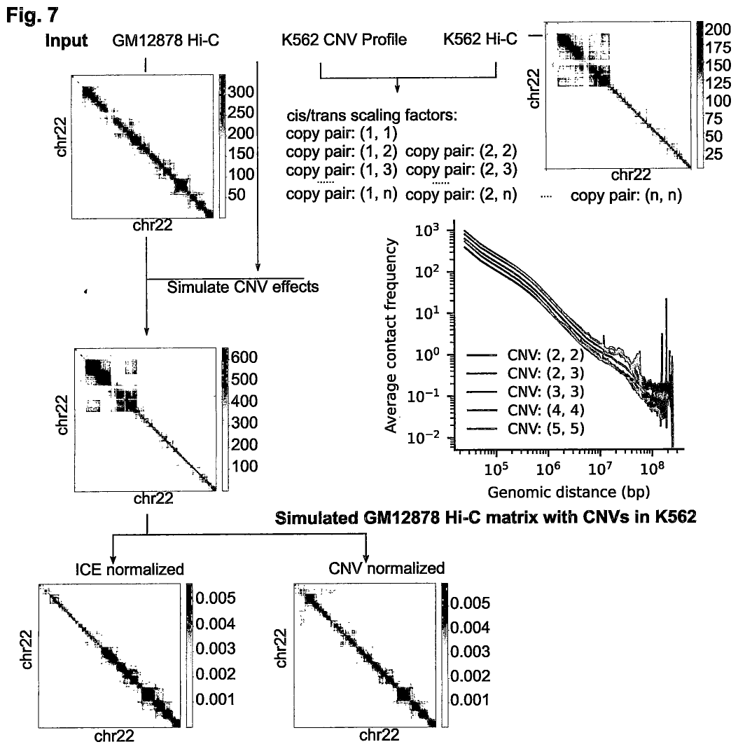

The method proposes a mathematical framework to simulate the effect of abnormal karyotypes on a diploid Hi-C dataset in order to benchmark the performance of its CNV normalization algorithm and the traditional ICE procedure. As shown in FIG. 7A, the inputs to the framework are the Hi-C matrix of a normal/diploid cell (GM12878), the Hi-C matrix of a cancer cell (K562) and the CNV profile of the same cancer cell. A scaling factor is estimated for both intra-chromosomal and inter-chromosomal contacts of different copy number regions in K562.

For inter-chromosomal contacts Mᵢⱼ^{trans}, the method calculates the average contact frequencies E_{(a,b)}^{trans} separately for different copy number pairs (a, b):

$${E}_{( a , b )}^{trans} = \frac{1}{n} \sum {M}_{ij}^{trans , ( a , b )}$$

where n is the number of contacts with one locus having a genomic copies and the other loci having b genomic copies. Then for each copy number pair (a, b), the method calculates the linear factor F_{(a,b)}^{trans} by applying

F_{(a,b)}^{trans}=E_{(a,b)}^{trans}/E_{(2,2)}^{trans}

For intra-chromosomal contacts Mᵢⱼ^{cis}, the method first calculates the contact-distance decay curves for each copy number pair:

$${E}_{d}^{( a , b )} = \frac{1}{n} {\sum }_{\vert i - j \vert = d} {M}_{ij}^{cis , ( a , b )}$$

After validating the linear relationship between curves from different copy number regions, the method establishes a linear regression model to estimate the scaling factor F_{(a,b)}^{cis} as follows:

E_{d}^{(a,b)}=F_{(a,b)}^{cis}·E_{d}^{(2,2)}+ε

To simulate the same CNV effects in GM12878, the K562 copy number profiles are imposed to GM12878 in silico. For any contact with virtual copy number (a, b), the value is multiplied by either F_{(a,b)}^{trans} or F_{(a,b)}^{cis}. It has been found that the resulted Hi-C map canreproduce aberrant signals in K562 within the same regions.

### d) Rearranged Fragment Detection, SV Filtering and Complex SV Assembling

Cancer genomes are shaped with both simple SVs and complex SVs. An algorithm developed in a previous work has been able to identify various kinds of simple SVs using Hi-C (Dixon, J. R. et al., Nat Genet 50, 1388 (2018)). The algorithms proposed in this method demonstrates that Hi-C can also be used in assembling complex SVs.

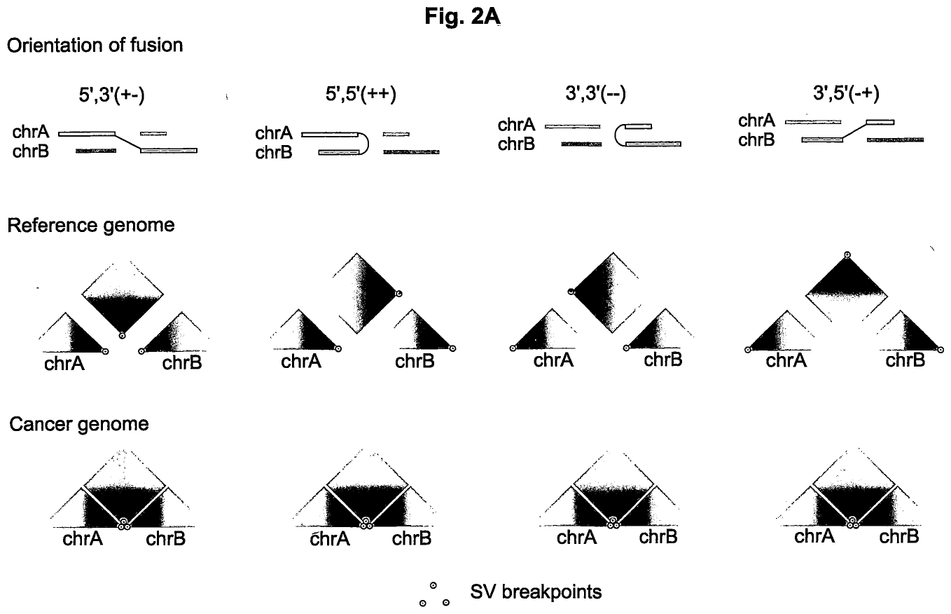

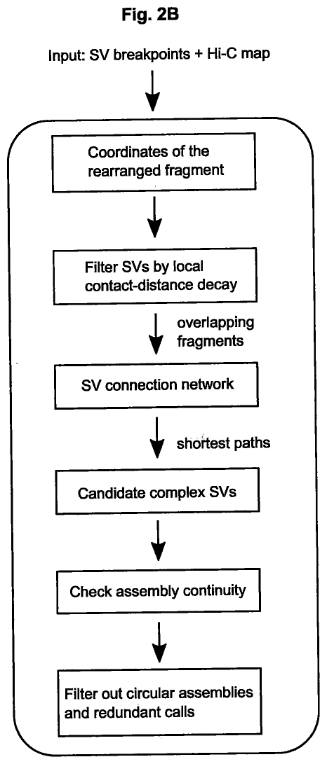

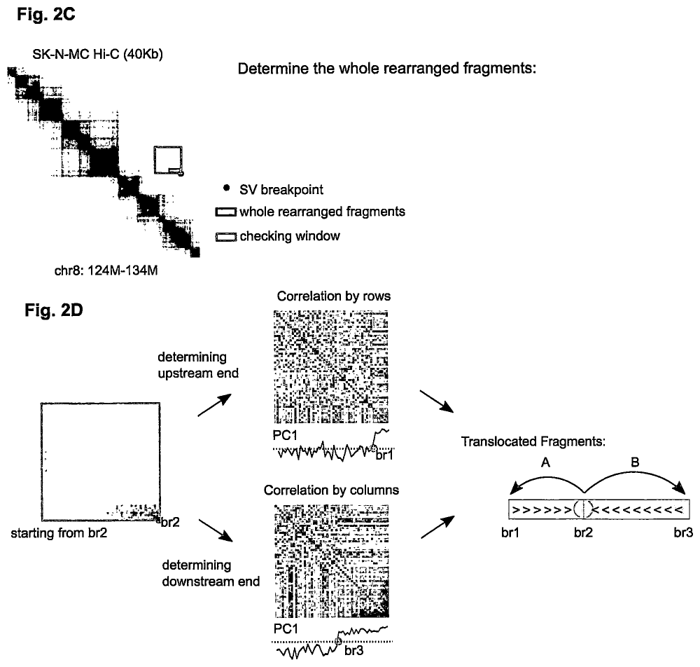

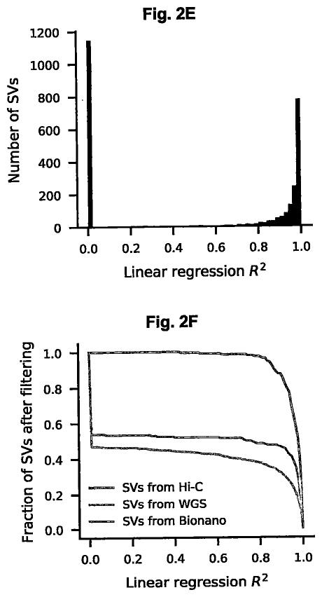

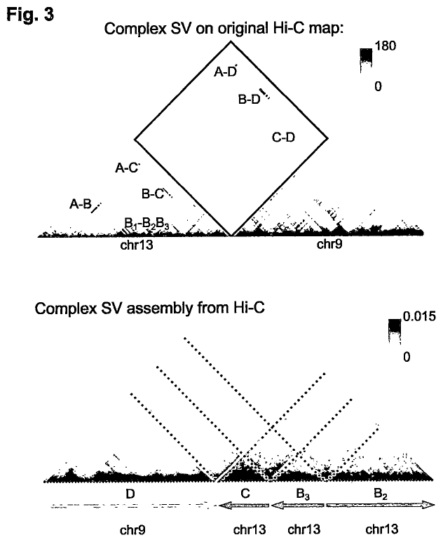

The input to the algorithm is a list of simple SV breakpoints and the genome-wide Hi-C matrix (FIG. 2B). Only large SVs are supported, including intra-chromosomal rearrangements greater than 1 Mb and inter-chromosomal translocations, which could be identified from various platforms such as Hi-C, WGS and optical mapping. The input SV coordinates are the coordinates of the breakpoints with strand information. The coordinates of the other end of junction fragments are still unknown. To determine whether a chain of SVs should be called complex SVs, the method begins with the detection of the coordinates of the whole chromatin fragments that are rearranged by each SV. To do so, the interaction region Mᵢⱼⁱⁿⁱ from breakpoints is extended by 5 Mb (this parameter is configurable) in appropriate orientations (the gray box in FIGS. 2C-D). Then two correlation matrices by rows and columns of Mᵢⱼⁱⁿⁱ are calculated respectively. As shown in FIG. 2D, the stretch of the rearranged fragments can be recognized by locating the corner square block on the correlation matrix. The principal component analysis is therefore performed, and the boundary loci are identified where the sign of the first eigenvector/principal component changes. Within Mᵢⱼⁱⁿⁱ , the region encompassed by the detected boundaries is denoted as Mᵢⱼ^{induced} (the green box in FIG. 2C).

Next the method filters out SVs from further consideration if no significant contact-distance decay can be dined detected within Mᵢⱼ^{induced}. Here the method employs a similar rationale used in Hi-C guided genome assembly: Hi-C signals should be continuous and attenuated along with the increasing genomic distance (contact-distance decay) across all regions on a valid SV assembly. Specifically, the method first calculates the global average contact frequencies E_{d}^{G} among all contact pairs with the same genomic distance throughout the whole genome:

$${E}_{d}^{G} = \frac{1}{n ( d )} {\sum }_{\vert i - j \vert = d} {M}_{i j}^{G}$$

where Mᵢⱼ^{G} denotes the whole-genome contact matrix, and n(d) denotes the number of valid data points at the genomic distance d. Similarly, the local distance averaged contact frequencies E_{d}^{induced} within Mᵢⱼ^{induced} is calculated as follows:

$${E}_{d}^{induced} = \frac{1}{n ( d )} {\sum }_{\vert i - j \vert = d} {M}_{i j}^{induced}$$

Then a linear regression model is fitted between E_{d}^{induced} and E_{d}^{G} for each SV, and only SVs with R²>0.6 will be considered (FIGS. 2E-F).

Then the potential complex SVs are detected by checking the overlap of rearranged fragments between simple SVs. A directed graph is built with each node representing a simple SV, and each edge representing an overlap of rearranged fragments in consistent orientations. The shortest paths between any two nodes of the graph are calculated by the Dijkstra's algorithm and each such path is defined as a candidate complex SV. During this procedure, Hi-C signals are re-mapped to each candidate assembly and the same linear regression-based method described above is applied to determine the assembly continuity. In other words, the method makes ensure each region of an assembly displays a significant distance-decay trend. Certain candidates are removed if the whole path or part of the path form a circular assembly. Finally, redundant candidates are removed if their paths are a subset or a reverse of other paths.

### e) Allele Normalization

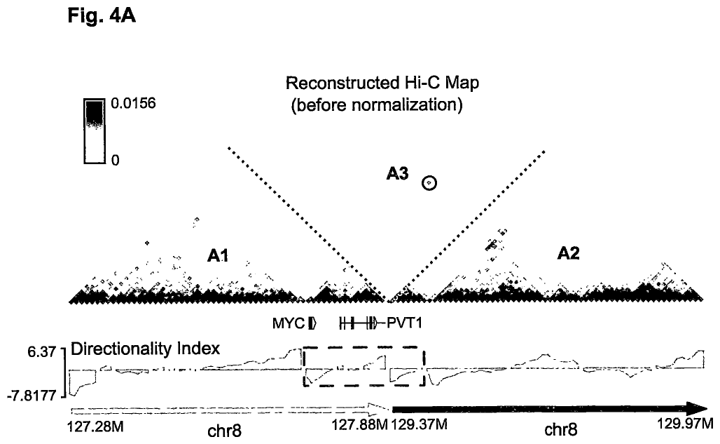

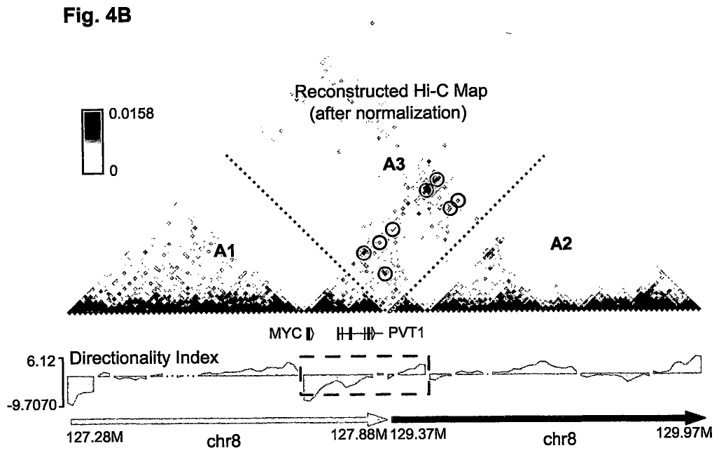

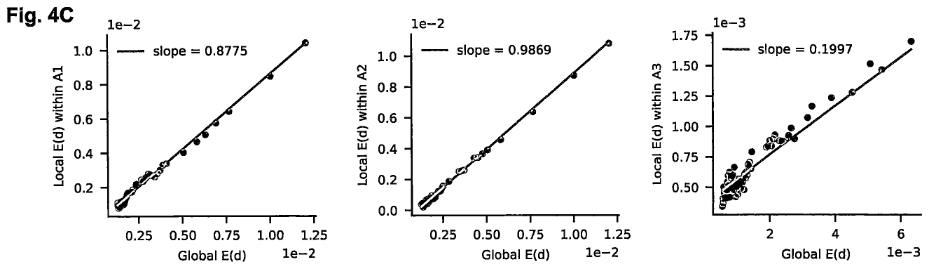

Due to heterogeneity of patient samples and heterozygosity of SVs, different regions in an assembly could have different Hi-C “visibility”. In other words, SVs that are less frequent in a sample and only occur within few alleles have a lower chance to be sequenced in the Hi-C experiment, which makes it difficult to accurately detect loops or TADs in these regions (FIG. 4A). Such biases can be captured by the distance decay curve between the contact frequencies and the genomic distance between the contact pairs. To remove the biases across different regions, a linear regression-based method is applied to minimize the differences between local distance decay curves and the whole-genome distance decay curve.

First the global average contact frequencies E_{d}^{G} is calculated using the same formula defined in the previous section. The maximum genomic distance considered was limited to 2 Mb. For an assembly composed of N chromatin fragments, the method calculates the local average contact frequencies E_{d}^{(a,b)} for each of the

$$C ( N , 2 ) = ( \begin{matrix}N \\ 2\end{matrix} )$$

contact regions between the fragment a (a≤N) and fragment b (b≤N). Any genomic distances with less than 3 non-zero data points is ignored and an isotonic regression method is applied to ensure the average contact frequencies are monotonically nonincreasing with the increasing genomic distance. Then the linear regression model is built as follows:

E_{d}^{(a,b)}=β^{(a,b)}·E_{d}^{G}+ε^{(a,b)}

where β^{(a,b)} denotes the estimated scaling factor. To make the model robust to outliers, the Huber loss function is utilized instead of the traditional least-squares. Finally, the contact frequencies Mᵢⱼ^{(a,b)} is rescaled by applying:

Aᵢⱼ^{(a,b)}=Mᵢⱼ^{(a,b)}/β^{(a,b)}

which potentially accounts for both sample heterogeneity and allelic effects.

### f) Machine-Learning Based Loop Detection

For loop detection, Peakachu, a recently developed machine-learning based framework (Salameh, T. J. et al., Nat Commun 11, 3428 (2020)) is utilized. Peakachu can learn the loop pattern on a genome-wide contact map from a set of known chromatin loops and then use the trained model to predict loops on other maps generated by the same experimental protocol. Cancer Hi-C datasets can be generated by different protocols, with varying sequencing depths and data quality. To build a general model, pre-trained models from the deeply sequenced GM12878 Hi-C library (Rao, S. S. P. et al., Cell 159, 1665-1680 (2014)) are included. Peakachu models are trained on both in-situ (Rao, S. S. P. et al., Cell 159, 1665-1680 (2014)) and dilution (Lieberman-Aiden, E. et al., Science 326, 289-293 (2009)) Hi-C map of the GM12878 cell. Both maps can be down-sampled to contain ˜60 million intra-chromosomal reads to improve the model sensitivity for a wider range of sequencing depths. For in-situ Hi-C, the model is trained for three resolutions: 10K, 20K, and 25K; for dilution Hi-C, the model is trained for five resolutions: 10K, 20K, 25K, 40K and 50K. The positive training sets are defined from the published CTCF ChIA-PET and H3K27ac HiChIP interactions, representing chromatin structural loops and regulatory loops, respectively.

In the prediction stage, the higher probability score computed by the CTCF or the H3K27ac model is recorded for each pixel of the reconstructed Hi-C map. After filtering with a pre-defined probability threshold, the same pooling algorithm used in Peakachu is applied to each SV region independently to select the best-scored loop contacts from each cluster. Loop lists from different resolutions are combined in a way that excluded redundant lower resolution loops. If a pixel is detected as a loop in both resolutions, the more precise location in higher resolution is recorded and the lower resolution one is discarded.

### g) Neo-TAD Identification

Neo-TAD detection algorithm is based on the directionality index (DI) and takes two steps:

The first step corresponds to the training stage of a traditional DI method, where DI is calculated along the reference genome (hg38) and used as input to learn a global HMM model. The DI is defined as a t-statistic as follows (Wang, X. T., Cui, W. & Peng, C., Nucleic Acids Res 45, e163 (2017)):

$${DI}_{i} = \frac{\frac{1}{W} {\sum }_{k = 1}^{W} {U}_{k} - \frac{1}{W} {\sum }_{k = 1}^{W} {D}_{k}}{\sqrt{\frac{{\sum }_{k = 1}^{W} {( {U}_{k} - \overline{{U}_{i}} )}^{2} + {\sum }_{k = 1}^{W} {( {D}_{k} - \overline{{D}_{i}} )}^{2}}{W ( W - 1 )}}}$$

where Uₖ denotes the upstream contact frequency between bin i and bin i−k, Dₖ denotes the downstream contact frequency between bin i and bin i+k, and W denotes the fixed window size which is set to 2 Mb in this study. A four-state (start, upstream bias, downstream bias and end) GMM-HMM model is defined by the pomegranate package and each state is emitted from a three-component Gaussian mixture. Again, the Baum-Welch algorithm is used for training.

In the second step, DI is recalculated on the allele normalized Hi-C map of each local assembly and the model trained above and the Viterbi algorithm are used to predict the state of each bin. The TADs are then defined as intervals between a “start” state and the next “end” state. And if a TAD has SV breakpoints located within its interval, it is called a neo-TAD.

### h) Visualization of Reconstructed Hi-C map and Genome Browser Tracks

The method introduces a standalone browser tool, specifically designed for visualizing shuffled cancer genome in the correct order. The tool supports plotting most kinds of tracks in a modern web browser, including genes, 1D peaks (.bed), and signals of RNA-Seq, ChIP-Seq, ATAC-Seq or DNase-Seq (.bigwig), 2D contact matrix (.cool) loops and TADs (.bedpe), etc.

When initializing a plotting object, the required input information is a cool URI and an individual line of the file outputted by the complex SV assembler, and both the number of tracks and the height of each track can be configured flexibly. For additional data types, miscellaneous methods are provided, which take regular bigwig/bed/bedpe files as input and automatically perform the coordinate conversion from the reference genome (hg38) to the local assembly in the correct orientations.

The method uses the cooler package for fast Hi-C data retrieval (Abdennur, N. & Mirny, L. A., Bioinformatics 36, 311-316 (2020)), the pyensembl package (https://github.com/openvax/pyensembl) to extract the gene information of any given regions, the CoolBox to draw genes in different styles (Xu, W. et al., bioRxiv, 614222 (2019)), and the pyBigWig for bigwig file loading (Ramirez, F. et al., Nucleic Acids Res 44, W160-165 (2016)). Most elements in each track are configurable, such as the colormap, value range of the Hi-C heatmap, the line color and style of the breakpoints, loop size and color, color and width of chromosome bars, the font and positions of gene names. The module is able to generate publication-quality figures, which can be conveniently saved to various image formats such as PNG, PDF, and SVG.

### Loop Detection Using FitHiC2

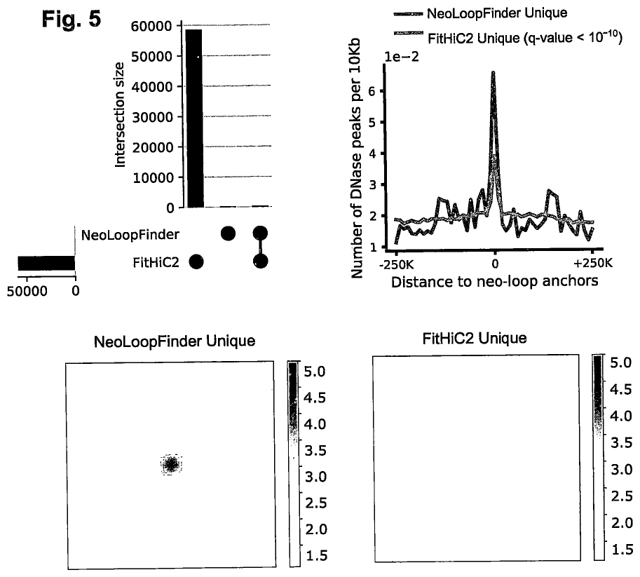

To investigate whether existing loop detection tools can be directly used to detect neo-loops without considering SV information, FitHiC2 (Kaul, A., Bhattacharyya, S. & Ay, F., Nat Protoc 15, 991-1012, (2020)) (fithic 2.0.7) (FIG. 5) has been tested for the purpose of loop detection. FitHiC2 is the only software that is able to detect inter-chromosomal interactions, as inter-chromosomal translocations happen frequently in cancer. FitHiC2 was run in all in situ Hi-C datasets analyzed in this invention at 10 kb resolution. For intra-chromosomal interactions, the results of the 2nd spline pass were first filtered with the q-value cutoff<10-10 and then the nearby significant interactions were pooled using the script “CombineNearbyInteraction.py” (https://github.com/ay-lab/fithic/tree/master/fithic/utils) with default parameters. For inter-chromosomal interactions, FitHiC2 was run in “-x interOnly” mode and applied a q-value cutoff of 10-10 for filtering. Both inter-chromosomal and intra-chromosomal interactions identified by FitHiC2 were mapped to SV assemblies reconstructed by this computational framework for comparison.

This pipeline has been applied to 50 Hi-C datasets from cancer cell lines and patient samples spanning 17 cancer types. Distinct sets of neo-loop-involved genes have been identified in different cancer types, which are enriched in cancer type-specific pathways and closely related to patient survival. By integrating H3K27ac ChIP Seq, chromatin accessibility (DNase-Seq), and RNA-Seq data from the ENCODE Consortium, this method further annotates enhancer hijacking events within the neo-55 loops, and validates them by CRISPR genome editing experiments.

### USES AND APPLICATIONS

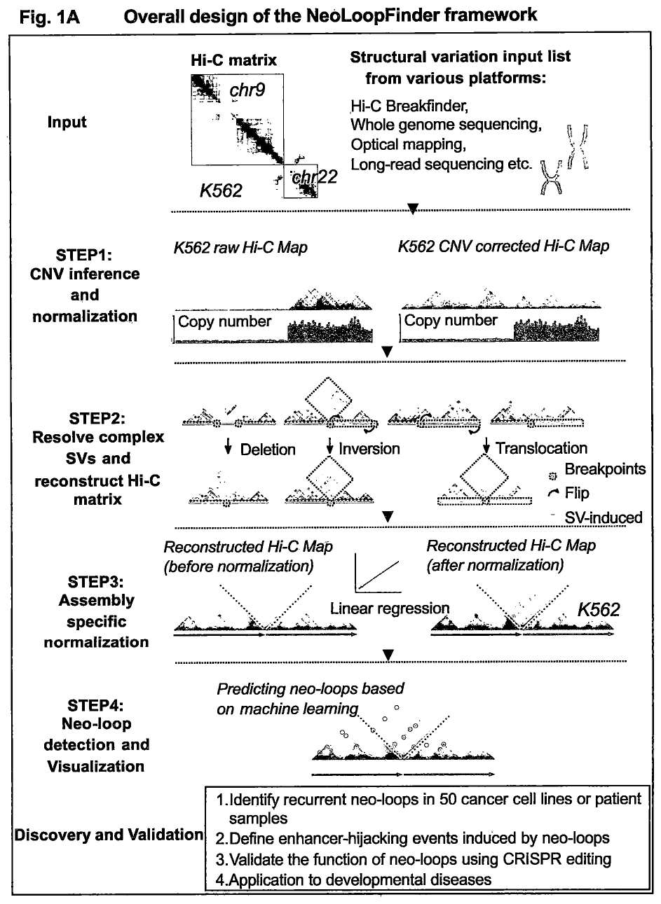

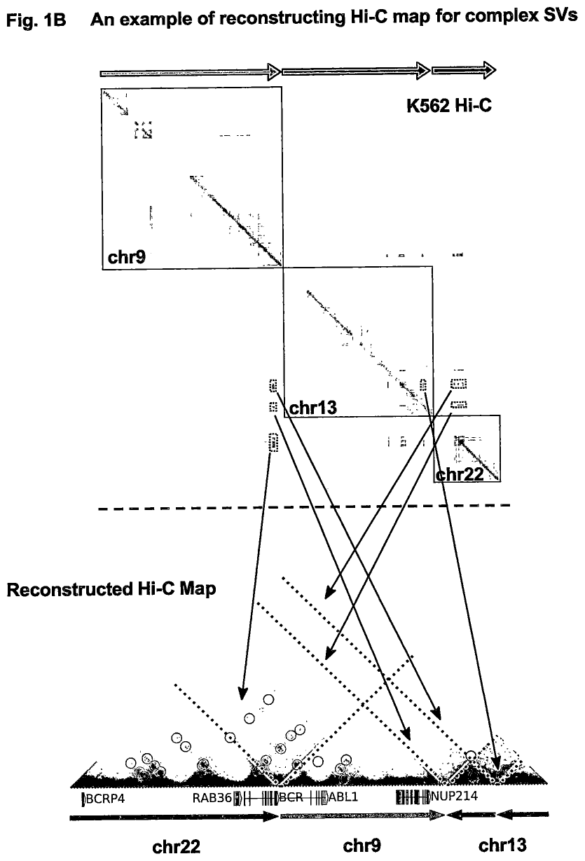

The methods described herein can be used to infer and normalize CNVs. As copy number variations (CNVs) can distort Hi-C signals in cancer cells (STEP 1 in FIG. 1A), a modified matrix balancing algorithm implemented in the methods can remove such effects along with other systematic biases including mappability, GC content, and restriction fragment sizes.

The methods can be used to resolve complex SVs and reconstruct Hi-C matrix. The methods can assemble the correct order of each individual SVs (FIG. 1B and FIGS. 2B-F). The methods can determine the other endpoint of the DNA fragments affected by SVs, and build a connection network to connect the rearranged fragments and resolve the complex SVs. The continuity of an output assembly is guaranteed by comparing local contact-distance decay with the global contact-distance decay pattern.

The methods can be used to normalize allele effects. Due to the heterozygosity of SVs and potential heterogeneity of patient samples, Hi-C signal intensity across the breakpoints tends to be lower than signals in the genomes not affected by SVs. The methods are able to scale signals from different regions into a similar range (STEP 3 in FIG. 1A and FIGS. 4A-C).

The methods can be integrated with machine-learning based framework referred to as Peakachu (STEP 4 in FIG. 1A) (Salameh, T. J. et al., Nat Commun 11, 3428 (2020)). The methods can train models at various resolutions and window sizes, covering both in situ and dilution Hi-C protocols to best handle the variable sequencing depths and data quality in different samples. The methods can identify loops in the regions not affected by SVs and chromatin loops induced by SVs, which are referred to as neo-loops. These neo-loops will serve as the basis for the detection of enhancer hijacking events.

The methods encompass a visualization module to facilitate the integration with other omics data, which provides functions such as plotting triangular contact heatmaps, genes, epigenomic tracks, and loops, using SV assembly coordinates with correct orientations.

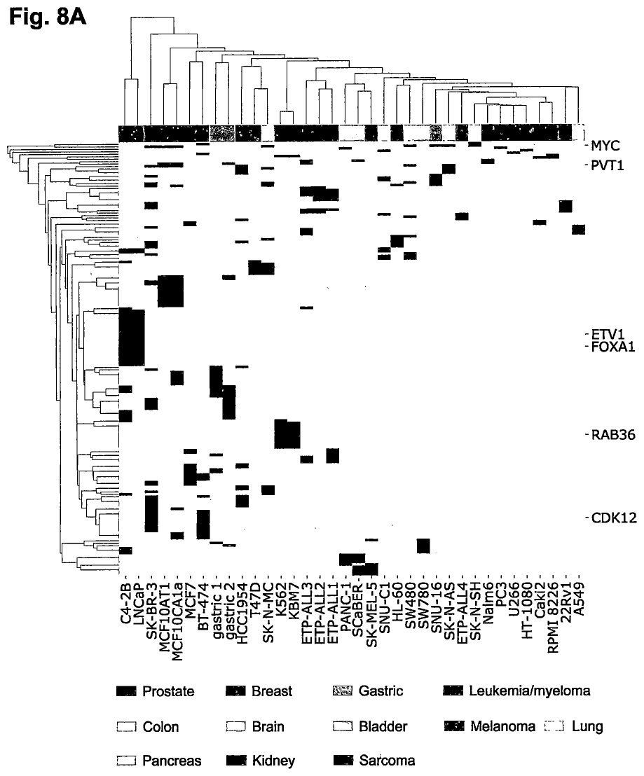

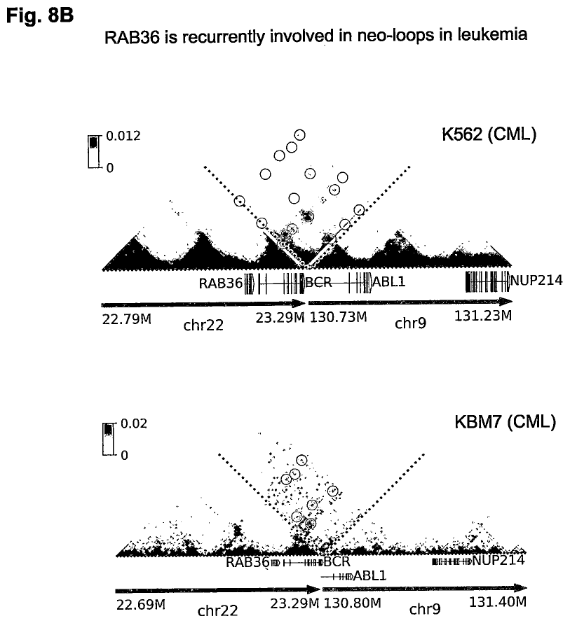

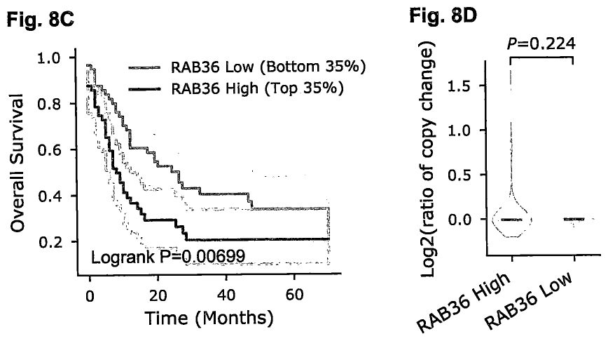

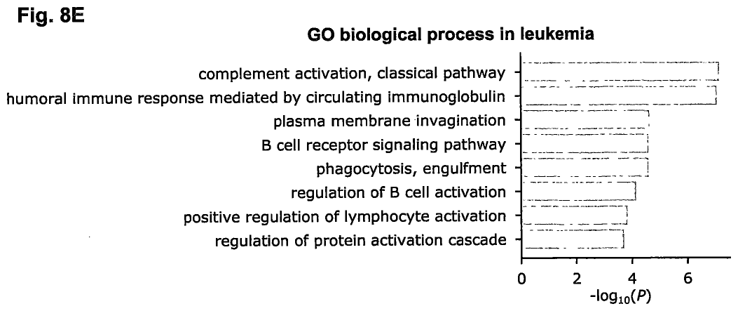

The methods can be used to discover novel cancer type-specific genes by neo-loops. In one example, 3,459 genes within neo-loops across 17 different cancer types have been identified. For ˜89.6% (3,099/3,459) of them, their gene bodies are not disrupted by SV breakpoints. Besides MYC and ETV1, many other known cancer-related genes have also been recognized, such as PVT1 (in SK-BR-3, MCF10AT, MCF10CA1, HCC1954, SK-N-AS, and SW480), a non-coding gene that regulates proliferation in a wide range of cancers (Derderian, C., Orunmuyi, A. T., Olapade-Olaopa, E. O. & Ogunwobi, O. O., Front Oncol 9, 502 (2019)); CDK12 (in SK-BR-3 and BT-474), a therapeutic target in triple-negative breast cancer (Quereda, V. et al., Cancer Cell 36, 545-558 e547 (2019)); FOXA1 (in C4-2B and LNCaP), a pioneer transcription factor that is frequently mutated in hormone-receptor-driven tumors, and has been linked to enhancer hijacking in metastatic prostate cancers (Parolia, A. et al., Nature 571, 413-418 (2019)). Overall, ˜8.0% (247/3,099) of these genes have been reported as cancer-related genes and 3.8% (117/3,099) have been identified as enhancer-hijacking genes by CESAM or PANGEA from different patient cohorts (Weischenfeldt, J. et al., Nat Genet 49, 65-74 (2017); He, B. et al., Sci Adv 6, eaba3064 (2020)). Importantly, it has been found that samples of the same cancer type tend to be clustered together based on the occurrence of these neo-loop-involved genes in each sample (FIG. 8A). There is a total of 99 recurrent neo-loop-involved genes from different cancer types (same gene appears in Ñ 2 samples of the same cancer type). 19.2% of them (19/99) are located in the recurrent neo-loops.

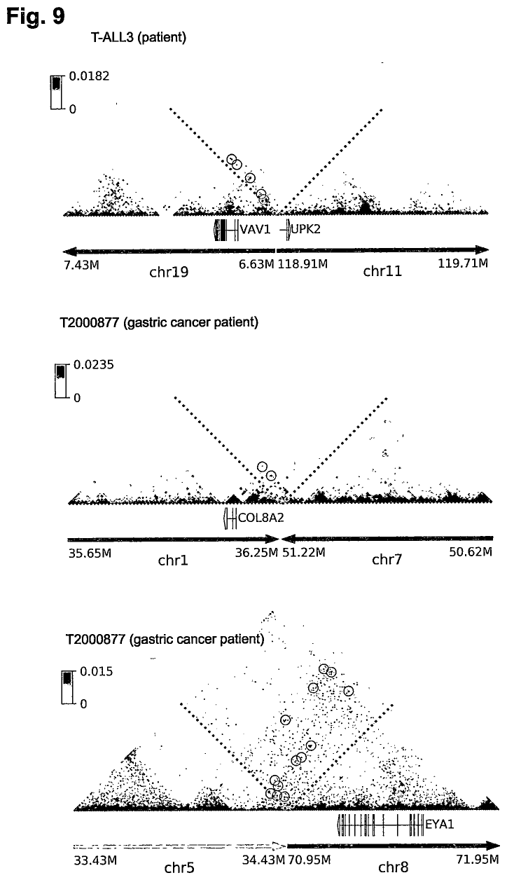

The methods are also able to report tens of novel genes that are non-fusion, unamplified, but might be involved in tumor proliferation or progression in addition to known cancer-related genes. In one example, it has been found that the RAB36 gene is located inside predicted neo-loops in two chronic myeloid leukemia cell lines (K562 and KBM7) (FIG. 8B). Notably, the overexpression of RAB36 is significantly associated with poorer overall survival across all available leukemia data from TCGA (P=0.00699, Log-rank test, FIG. 8C), and its overexpression cannot be explained by copy number variations in corresponding patients (FIG. 8D). These results indicate a prognostic role of RAB36 in leukemia and highlight enhancer hijacking as a potential regulatory mechanism. Similarly, it has been observed UPK2 in T-ALL and EYA1 in gastric cancer are involved in neo-loops (FIG. 9).

The methods can be used to indicate that neo-loop-involved genes are enriched in both tumorigenesis-associated and tissue-specific processes (FIG. 8E) (Kuleshov, M. V. et al., Nucleic Acids Res 44, W90-97 (2016)). For example, the most significant processes involved in leukemia are related to the immune system such as the classical complement pathway, the B cell receptor signaling pathway, and the regulation of lymphocyte activation. In breast cancer, genes within neo-loops are mostly enriched in the regulation of histone methylation, including H3K4 methylation, whose elevated level has been associated with a poor clinical outcome in various cancers (Spangle, J. M. et al., Cell Rep 15, 2692-2704 (2016)). In gastric cancers, it has been found neo-loops involved genes in both the regulation of epithelial cell proliferation and the regulation of insulin receptor signaling pathway.

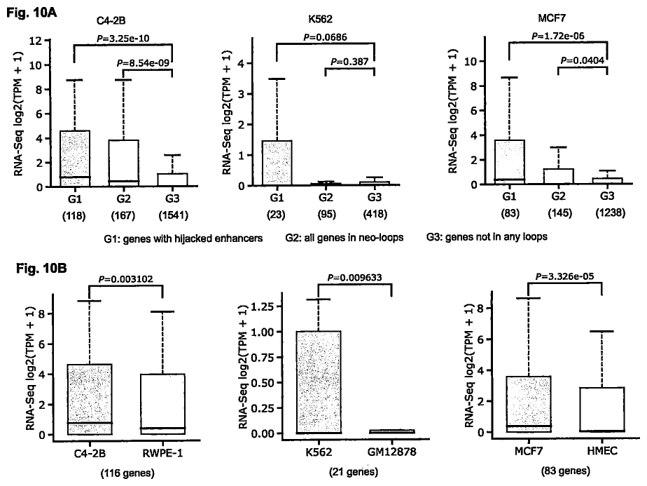

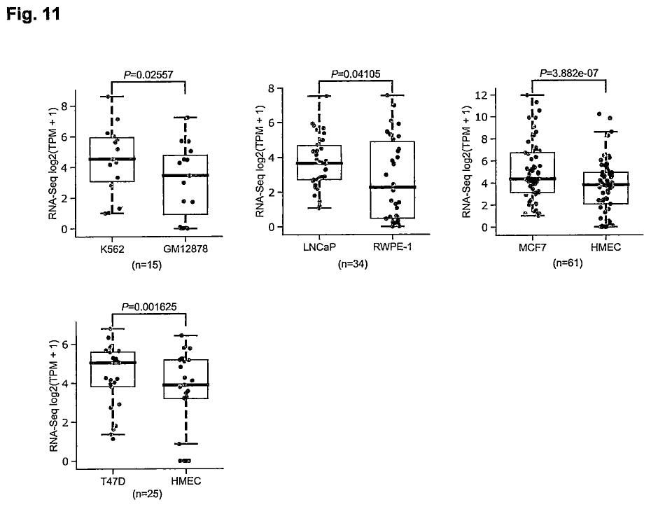

The methods can be used to detect enhancer hijacking events induced by neo-loops. To identify genes with hijacked enhancers, H3K27ac ChIP-Seq, chromatin accessibility (DNase-Seq), and RNA-Seq data from the ENCODE Consortium have been integrated. Potential enhancers are defined as H3K27ac or DNase-Seq peaks, and enhancer hijacking is defined as the following: a gene promoter located in one anchor of a predicted neo-loop, and there is an enhancer located in the other anchor of the neo-loop. It has predicted the enhancer-hijacking events in 11 cell lines, including A549, K562, LNCaP, MCF7, T47D, HepG2, SK-MEL-5, NCI-H460, PANC-1, HT-1080 and C4-2B. The genes involved in enhancer-hijacking events have shown higher expression compared with other neo loop-involved genes (FIG. 10A). These genes have also upregulated compared with their expression in control cell lines from the same tissue, further suggesting the role of enhancer-hijacking in gene dysregulation in cancer (FIG. 10B and FIG. 11).

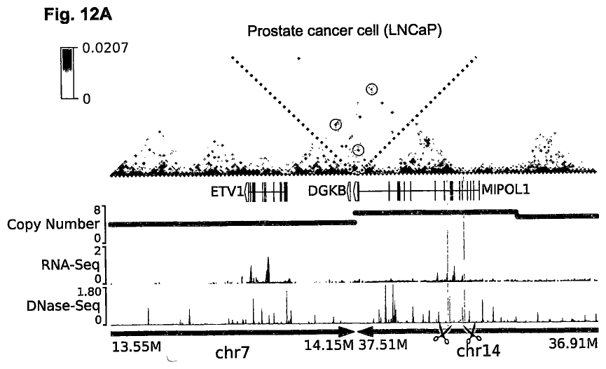

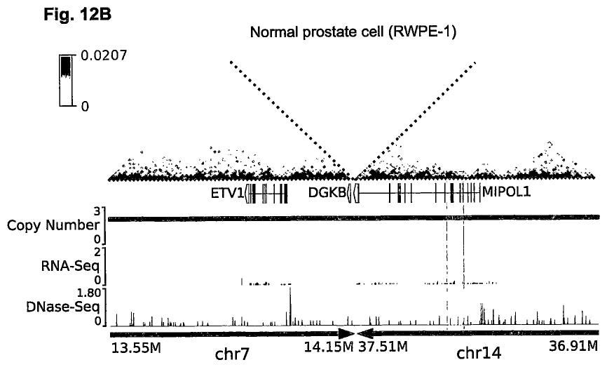

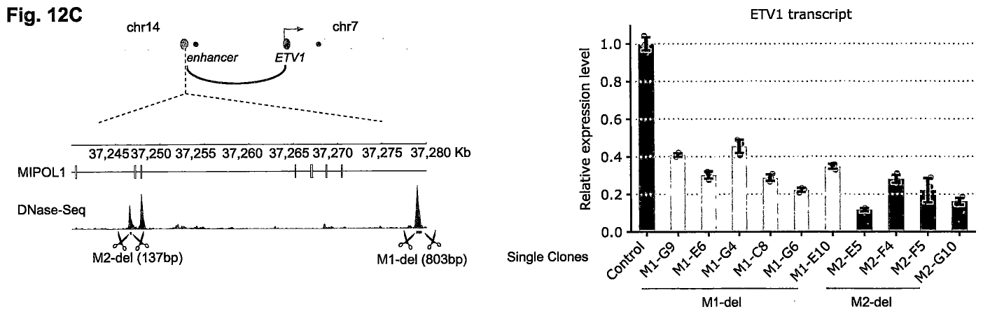

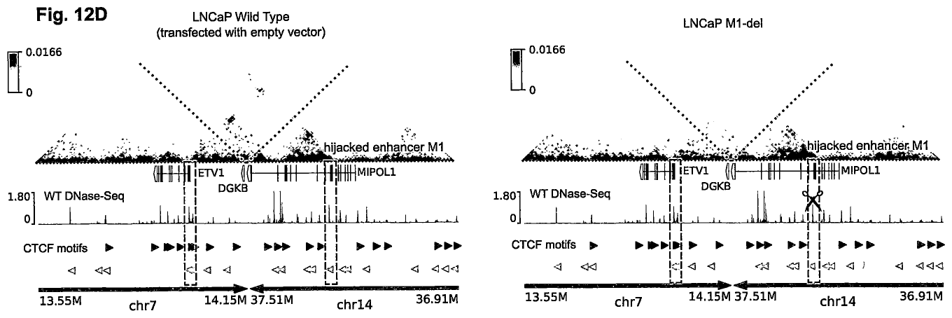

The methods can be used to help delete hijacked enhancers to reduce oncogene expression. In one example, genome editing experiment has been performed utilizing CRSPR/Cas9 system. ETV1 is a known driver gene in prostate cancer, whose over-expression by amplification or gene-fusion has been associated with aggressive disease and poorer outcomes (Baena, E. et al., Gene Dev 27, 683-698 (2013); Gasi, D. et al., Plos One 6, e16332, (2011)). It is observed that ETV1 is dramatically up-regulated in LNCaP (a prostate adenocarcinoma cell line), compared with normal prostate epithelial cells RWPE-1 (>16-fold) (FIGS. 12A-B). However, no gene fusion or amplification of ETV1 in LNCaP is observed. Rather, it is found that ETV1 is located inside neo-loops induced by a translocation between chromosome 7 and chromosome 14 (chr7: 14.15M, +; chr14: 37.51M, +), interacting with multiple potential enhancers on chromosome 14 (FIG. 12A, DNase-Seq, purple track). Moreover, it is observed that these neo-loops coincide with convergent CTCF binding motifs (FIG. 12D). As a comparison, there is no interplay between ETV1 and the same regions in RWPE-1 cells (FIG. 12B), suggesting that ETV1 overexpression in LNCaP might be driven by enhancer hijacking.

To validate the prediction, multiple CRISPR/Cas9 genome editing experiments are performed to delete the hijacked enhancers in these loci (FIG. 12A, yellow band; FIG. 12C). Within the neo-loop anchor, there are three DNase-Seq peaks, and one of them overlaps with a MIPOL1 exon. Only the two intronic DNase-Seq peaks (M1-del and M2-del) are deleted to rule out the potential effect of affecting the MIPOL1 gene. Then RT-qPCR is performed to compare the ETV1 expressions between the enhancer-deleted LNCaP cells and the control LNCaP cells (transfected with empty vector). The experiments are performed across 6 and 4 independent clones for M1-del and M2-del, respectively. A dramatic reduction of ETV1 expression in the engineered LNCaP cells is observed, with an average of 66.3% reduction of expression in M1-del and an average 80.5% reduction in M2-del clones, suggesting a novel mechanism for ETV1 activation in prostate cancers by enhancer hijacking. To further investigate the structural role of these hijacked enhancers, in situ Hi-C for both control cells and the M1-deleted cells (M1-E10, FIG. 12C) is performed. Strikingly, it is observed that the Hi-C signals in the neo-loop are greatly reduced (FIG. 12D), while the local interactions surrounding MIPOL1 are strengthened. This suggests that the deletion of the hijacked enhancers might have changed the overall structure in this region.

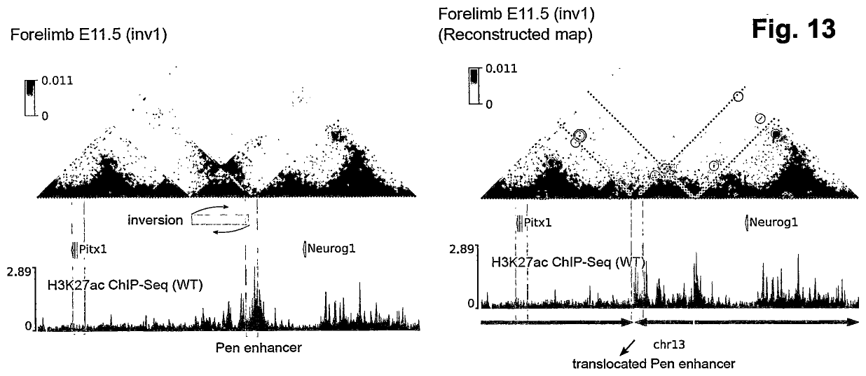

The methods can be used to detect enhancer hijacking in developmental diseases. In one example, data have been collected for various structural variations causing limb malformations that have been functionally dissected in previous studies: (1) an inversion involving an enhancer (named “Pen”) that controls the Pitx1 gene expression (Kragesteen, B. K. et al., Nat Genet 50, 1463-1473 (2018)); (2) a duplication covering the Sox9 regulatory domain and the Kcnj2 gene (Franke, M. et al., Nature 538, 265-269 (2016)); (3) an inversion of the Sox9 regulatory domain including the TAD boundary (Despang, A. et al., Nat Genet 51, 1263-1271 (2019)). As shown in FIG. 13, in each case, this computational framework has been able to automatically reconstruct the Capture Hi-C map for the local genome that is highly similar to the matrices presented in the original reports. Furthermore, this computational framework has also identified potential hijacked enhancer promoter interactions for the indicated disease-causal genes (Pitx1, Kcnj2, and Kcnj2). The proposed algorithm should work on any chromatin interaction data with rearranged genome for different types of SVs, such as inversion and duplications.

### DEFINITIONS

The term “structural variation” refers to the variation in structure of an organism's chromosome. It consists of many kinds of variation in the genome such as deletions, duplications, copy-number variants, insertions, inversions and translocations.

The term “copy number variation” refers a phenomenon when the number of repeats in the genome varies from one individual to the next.

The term “simple SVs” refers to assemblies that only contain two DNA fragments (or one junction).

The term “complex SVs” refers to local assemblies that are made up of multiple (≥3) DNA fragments (or ≥2 junctions) from different genomic locations on the reference genome.

The term “neo-loops” refers to chromatin loops induced by SVs. These neo-loops will serve as the basis for the detection of enhancer hijacking events.

The term “TAD” refers to intervals between a “start” state and the next “end” state. If a TAD has SV breakpoints located within its interval, it is called a neo-TAD.

The term “enhancer hijacking” refers to an event when there is a gene promoter located in one anchor of a predicted neo-loop, and there is an enhancer located in the other anchor of the neo-loop.

The term “Hi-C contact matrix” refers to a n-by-n matrix representing the interaction of loci or chromosomal regions as captured in the Hi-C experiment.

The term “SV breakpoints” refers to locations on a chromosome where structural variations occur.

All genomic datasets used in this work were mapped to the human genome assembly GRCh38 (hg38). Hi-C data were either downloaded from the 4DN data portal (https://data.4dnucleome.org/) or processed by the runHiC python package (https://pypi.org/project/runHiC/). The 472 CNV profile in each sample was directly estimated from Hi-C maps using a generalized additive model, and then an HMM based segmentation was applied to determine the boundaries of CNV segments. In FIGS. 6A-G, CNV profiles and segments from WGS reads were calculated by Control-FREEC (Boeva, V. et al., Bioinformatics 28, 423-425 (2012)) in 8 cell lines for the benchmark. The ploidy parameter was set as follows in running Control-FREEC: 2 for pseudodiploid cells SK-N-MC and hypotriploid cells NCI-H460; 3 for triploid cells K562, hypotriploid cells A549 and T47D, and hypertriploid cells PANC-1; 4 for hypotetraploid cells LNCaP; 5 for hypopentaploid or hypohexaploid cells Caki2. As another input to this computational framework, the breakpoints of large SVs were mostly identified from Hi-C maps using Hi-C break finder (https://github.com/dixonlab/hic_breakfinder), except for the aforementioned 8 cell lines A549, Caki2, K562, LNCaP, NCI-H460, PANC-1, SK-N-MC and T47D, where published SV lists compiled from multiple experimental platforms (Dixon, J. R. et al., Nat Genet 50, 1388 (2018)) were used. The RNA-Seq data were processed using the ENCODE long-read RNA-Seq pipeline (https://github.com/ENCODE-DCC/long-rna-seq-pipeline). Data used for survival analysis in leukemia and gastric cancer were downloaded from the cBioPortal for Cancer Genomics (https://www.cbioportal.org) (Liu, J. et al., Cell 173, 400-416 e411 (2018)). The list of cancer-related genes was obtained from the Bushman Lab (http://www.bushmanlab.org/assets/doc/allOnco_May2018.tsv).

### 4C-Seq Library Preparation and Sequencing.

4C experiments were performed in SK-N-MC cells (obtained from ATCC, https://www.atcc.org/) to quantify genome-wide DNA-DNA interactions that involve promoter of the MYC gene in this cell line. Ten million SK-N-MC cells in cell culture were harvested, resuspended in 8.75 ml 1× complete medium, and immediately cross-linked in 2% formaldehyde for 10 minutes. Nuclei were isolated from fixed cells and digested by restriction enzyme DpnII (NEB, #R0543) overnight. Digested DNA was ligated using T4 DNA ligase (NEB, #M2020) and de-crosslinked by incubating with proteinase K overnight (Qiagen, #19133). DNA was then purified and fragments larger than 30 kDa were selected and digested by the second restriction enzyme Csp6i (Thermo, #ER0211) overnight. DNA was re-ligated using DNA ligase (1U/ul, Invitrogen, #15224017) at low concentration. DNA was further purified and DNA fragments that contain MYC promoter were amplified by PCR reaction with 30 cycles: A transcription start site of MYC gene flanked by DpnII and Csp6i cutting site at each end was chosen as an anchor (chr8: 127,735,189-127,735,615), and a pair of primers that contain Truseq indices were used to amplify regions located to the upstream and downstream of the anchor. The PCR products were further purified by size selection to remove small DNA fragments, and sent for sequencing on Illumina HiSeq 2500 with a depth of 2 million reads.

The sequences of primers are as following:

MYC-upstream:
CAAGCAGAAGACGGCATACGAGAGATGTAGCCGTGACTGGAGTTCAGAC
GTGTGCTCTTCCGATCTGTTGCCTGCTCTCTGCCAGT
MYC-downstream:
AATGATACGGCGACCACCGAGATCTACACTCTTTCCCTACACGACGCTC
TTCCGATCTTCCACTCTCCCTGGGACTCT

For a computer readable form (CRF) of the sequence listing, please refer to the text file named “DNA-sequence.txt” on CD-ROM incorporated in this invention.

The 4C sequencing reads were first trimmed by 23 bp from the 5′ end to remove the bait sequence. Trimmed reads were then mapped to human reference GRCh38 with “BWA MEM”. Per-base read coverage was then calculated by “bedtools genomecov”.

### CRISPR/Cas9-Mediated Enhancer Deletion.

sgRNAs were designed by combining results from CRISPOR (http://crispor.tefor.net) and GuideScan (http://www.guidescan.com). Two pairs of sgRNA were chosen for each site. sgRNA sequences and PCR primers are listed in Table 1. The gRNA oligos were inserted into pX458 vectors (Addgene) using the BbsI restriction enzyme (NEB). For target deletion, the LNCaP cell line (obtained from ATCC, https://www.atcc.org/) was transfected with each pair of gRNA vector aiming to upstream and downstream of the target region (named M1 and M2) and flow-sorted for GFP positive cells after 2 days. Individual cells were seeded into 96-well plates and expanded for 3-4 weeks. Single-cell clones were selected and amplified. The deletion was verified by genomic DNA PCR with the primers outside the target deletion region and Sanger sequencing.

### RNA Extraction and Real-Time PCR.

RNA was extracted using RNeasy Mini kit (Qiagen) according to the manufacturer's instructions. For cDNA preparation and DNA elimination, the SuperScript® III First-Strand Synthesis System (Invitrogen) was used according to the manufacturer's instructions. qRT-PCR was performed on a Bio-Rad CFX Connect Real-time PCR Detection system using KAPA SYBR FAST qPCR Master Mix. PCR conditions were as follows: 1 cycle of 95° C. for 3 min and 40 cycles of 95° C. for 15 sec, 60° C. for 15 sec and 72° C. for The housekeeping gene GAPDH was used as a normalization control. The primer sequences are listed in Table 1.

TABLE 1
Oligos for gRNAs and real-time PCR

| Oligos | Sequences (5′-3′) | PAM |
|---|---|---|
| M1-gRNA1 | GTACGAGTGAGACAGTACCT | TGG |
| M1-gRNA2 | TGGGAGGATAGACATTTCAC | AGG |
| M1-gRNA3 | TTTGTTATAGTCATCTACCT | TGG |
| M1-gRNA4 | AGGTTAGATGTGAAATTTGT | TGG |
| M2-gRNA1 | AGTTATTCCCTTAAATGCCC | AGG |
| M2-gRNA2 | CTAGAGGCTTTCTAAAACTT | GGG |
| M2-gRNA3 | AAGCCATATCAGATCCTAGT | TGG |
| M2-gRNA4 | CAATCATTTAATTACACCTG | AGG |
| M1F-outside | TTTCCTTCTTTTTAATTTAGCC | |
| MIR-outside | AAAAACCCTCAAAACCAAAA | |
| M2F-outside | TCATTAATTATCTCAGTGTCCAT | |
| M2R-outside | CACTCAGAAAAGTAAACACCAA | |
| ETV1-qRT-F | ACTCCATTTATATGAGGCAAGAAG | |
| ETV1-qRT-R | TTGTTTGATGTCTCCATCGAAT | |

For a computer readable form (CRF) of the sequence listing, please refer to the text file named “SequenceListing.txt” submitted through the EFS-Web that is incorporated in this invention.

### Hi-C Library Preparation and Sequencing.

Approximately 2M of LNCaP wild type and LNCaP M1-deleted (M1-E10) cells were pelleted and crosslinked with 2% formaldehyde in PBS at RT for 10 mins. ARIMA's Hi-C kit (ARIMA Genomics) was applied for capturing proximally-ligated DNA. The biotinylated DNA was sheared to a size of 300-500 bp and libraries were constructed following Kapa Hyper Prep Kit (KAPA) protocols. The library products were quantified with Tapestation High Sensitivity D1000 Assay (Agilent Technologies, CA, USA) and then sequenced using Illumina's NovaSeq platform (Illumina).

### EXAMPLES

### Detection of Neo-Loops in 50 Cancer Samples.

In one example, the computational method was applied to 50 Hi-C datasets spanning 17 cancer types. Among them, SVs in 8 cell lines have been identified in the previous work (Dixon, J. R. et al., Nat Genet 1388-+(2018)). For other samples, the Hi-C breakfinder was run to define the SV breakpoints.

The Hi-C contact matrices were downloaded from the 4DN data portal (https://data.4dnucleome.org/) if the processed data were available in the portal. For other samples, the raw SRA data were downloaded from the GEO database and processed the data using the runHiC (https://pypi.org/project/runHiC/, v0.8.4) software.

The CNV calculation module from the computational method was applied to calculate the copy number variation profiles from Hi-C.

The CNV correction module from the computational method was applied to remove the copy number variation biases along with other systematic biases from Hi-C matrices.

For WGS data, the Control-FREEC (v11.6) was used to define copy number variation profiles in 8 cancer cell lines to evaluate the performance of the CNV calculation module from the computational method.

The local Hi-C maps were reconstructed with the complex SV assembly module from the computational method.

“Neo-loops” on the reassembled Hi-C map were predicted with the loop detection module which is based on the Peakachu framework (Salameh NC 2020)

The Hi-C maps and other genomic and epi-genomic tracks on cancer-specific local assemblies were visualized with the visualization module from the computational method.

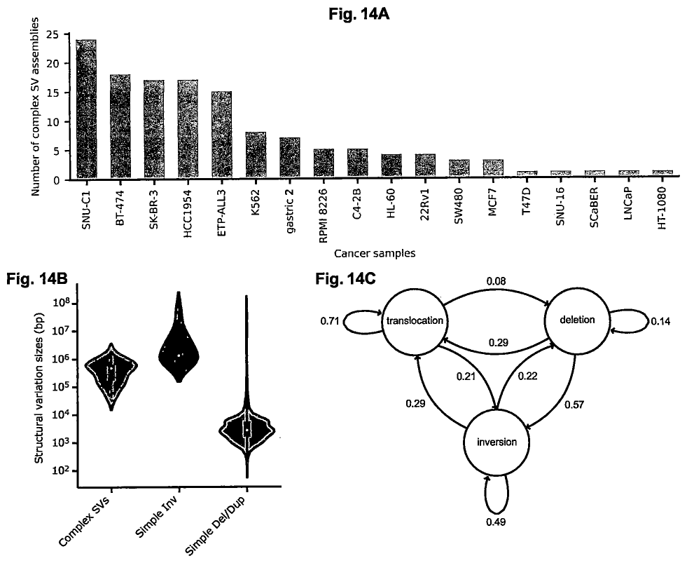

In total, 1,510 large SVs were collected across all samples. With the use of graph-based algorithm, complex SVs in 18 samples (FIGS. 14A-C) could be detected. The rearranged fragments in a complex SV are generally smaller than simple inversions but larger than simple deletions/duplications (FIG. 14B).

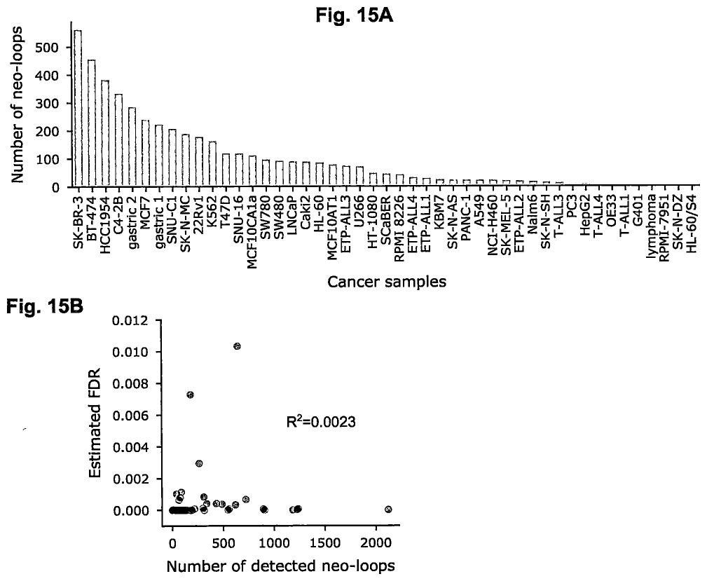

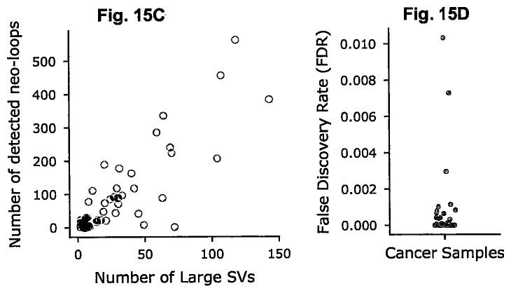

The number of neo-loops detected in each sample ranges from 0 to 562, with a median value of 37 (FIG. 15A). The number of neo-loops is approximately proportional to the number of SVs in each sample (FIG. 15C). To estimate the false discovery rate (FDR), the SV breakpoints were randomly shuffled 50 times for each dataset, controlling for the distribution of SV types, the ratio of inter- vs. intra-chromosomal SVs, and the sizes of the SVs. The estimated FDRs are less than 1% for 97.9% (46 out of 47 samples that have neo-loops detected) of the samples (FIG. 15D), and 59.6% (28/47) of them have an FDR of 0. Importantly, the estimated FDRs and the number of neo-loops in corresponding samples are not correlated with each other (FIG. 15B). Therefore, it was concluded that the neo-loop interactions detected in this study were not likely to be caused by data noise.

### REFERENCES

1 Futreal, P. A. et al. A census of human cancer genes. Nat Rev Cancer 4, 177-183 (2004).
2. Beroukhim, R., Zhang, X. Y. & Meyerson, M. Copy number alterations unmasked as enhancer hijackers. Nat Genet 49, 5-6 (2017).
3. Consortium, E. P. An integrated encyclopedia of DNA elements in the human genome. Nature 489, 57-74 (2012).
4. Bernstein, B. E. et al. The NIH Roadmap Epigenomics Mapping Consortium. Nat Biotechnol 28, 1045-1048 (2010).
5. Weischenfeldt, J. et al. Pan-cancer analysis of somatic copy-number alterations implicates IRS4 and IGF2 in enhancer hijacking. Nat Genet 49, 65-74 (2017).
6. Spielmann, M., Lupianez, D. G. & Mundlos, S. Structural variation in the 3D genome. Nat Rev Genet 19, 453-467 (2018).
7 Groschel, S. et al. A Single Oncogenic Enhancer Rearrangement Causes Concomitant EVI1 and GATA2 Deregulation in Leukemia. Cell 157, 369-381 (2014).
8. Drier, Y. et al. An oncogenic MYB feedback loop drives alternate cell fates in adenoid cystic carcinoma. Nat Genet 48, 265-272 (2016).
9. Yang, M. et al. 13q12.2 deletions in acute lymphoblastic leukemia lead to upregulation of FLT3 through enhancer hijacking. Blood (2020).
10. Ooi, W. F. et al. Integrated paired-end enhancer profiling and whole-genome sequencing reveals recurrent CCNE1 and IGF2 enhancer hijacking in primary gastric adenocarcinoma. Gut 69, 1039-1052 (2020).
11. Martin-Garcia, D. et al. CCND2 and CCND3 hijack immunoglobulin light-chain enhancers in cyclin D1(−) mantle cell lymphoma. Blood 133, 940-951 (2019).
12. Haller, F. et al Enhancer hijacking activates oncogenic transcription factor NR4A3 in acinic cell carcinomas of the salivary glands. Nat Commun 10, 368 (2019).
13. Zimmerman, M. W. et al. MYC Drives a Subset of High-Risk Pediatric Neuroblastomas and Is Activated through Mechanisms Including Enhancer Hijacking and Focal Enhancer Amplification. Cancer Discov 8, 320-335 (2018).
14. Ryan, R. J. et al. Detection of Enhancer-Associated Rearrangements Reveals Mechanisms of Oncogene Dysregulation in B-cell Lymphoma. Cancer Discov 5, 1058-1071 (2015).
15. Northcott, P. A. et al Enhancer hijacking activates GFI1 family oncogenes in medulloblastoma. Nature 511, 428-434 (2014).
16. He, B. et al. Diverse noncoding mutations contribute to deregulation of cis-regulatory landscape in pediatric cancers. Sci Adv 6, eaba3064 (2020).
17. Franke, M. et al. Formation of new chromatin domains determines pathogenicity of genomic duplications. Nature 538, 265-269 (2016).
18. Northcott, P. A. et al. The whole-genome landscape of medulloblastoma subtypes. Nature 547, 311-317 (2017).
19. Lieberman-Aiden, E. et al. Comprehensive Mapping of Long-Range Interactions Reveals Folding Principles of the Human Genome. Science 326, 289-293 (2009).
20. Wang, S. et al. HiNT: a computational method for detecting copy number variations and translocations from Hi-C data. Genome Biol 21, 73 (2020).
21. Dixon, J. R. et al. Integrative detection and analysis of structural variation in cancer genomes. Nat Genet 50, 1388-+(2018).
22. Chakraborty, A. & Ay, F. Identification of copy number variations and translocations in cancer cells from Hi-C data. Bioinformatics 34, 338-345 (2018).
23. Rao, S. S. P. et al. A 3D Map of the Human Genome at Kilobase Resolution Reveals Principles of Chromatin Looping. Cell 159, 1665-1680 (2014).
24. Imakaev, M. et al. Iterative correction of Hi-C data reveals hallmarks of chromosome organization. Nat Methods 9, 999-1003 (2012).
25. Wu, H. J. & Michor, F. A computational strategy to adjust for copy number in tumor Hi-C data. Bioinformatics 32, 3695-3701 (2016).
26. Vidal, E. et al. OneD: increasing reproducibility of Hi-C samples with abnormal karyotypes. Nucleic Acids Res 46, e49 (2018).
27. Servant, N., Varoquaux, N., Heard, E., Barillot, E. & Vert, J. P. Effective normalization for copy number variation in Hi-C data. BMC Bioinformatics 19, 313 (2018).
28. Salameh, T. J. et al. A supervised learning framework for chromatin loop detection in genome-wide contact maps. Nat Commun 11, 3428 (2020).
29. Fudenberg, G. et al. Formation of Chromosomal Domains by Loop Extrusion. Cell Rep 15, 2038-2049 (2016).
30. Sanborn, A. L. et al. Chromatin extrusion explains key features of loop and domain formation in wild-type and engineered genomes. Proc Natl Acad Sci USA 112, E6456-6465 (2015).
31. Crane, E. et al. Condensin-driven remodelling of X chromosome topology during dosage compensation. Nature 523, 240-244 (2015).
32. Dixon, J. R. et al. Topological domains in mammalian genomes identified by analysis of chromatin interactions. Nature 485, 376-380 (2012).
33. Derderian, C., Orunmuyi, A. T., Olapade-Olaopa, E. O. & Ogunwobi, O. O. PVT1 Signaling Is a Mediator of Cancer Progression. Front Oncol 9, 502 (2019).
34. Quereda, V. et al. Therapeutic Targeting of CDK12/CDK13 in Triple-Negative Breast Cancer. Cancer Cell 36, 545-558 e547 (2019).
35. Parolia, A. et al. Distinct structural classes of activating FOXA1 alterations in advanced prostate cancer. Nature 571, 413-418 (2019).
36. Spangle, J. M. et al. PI3K/AKT Signaling Regulates H3K4 Methylation in Breast Cancer. Cell Rep 15, 2692-2704 (2016).
37. Baena, E. et al. ETV1 directs androgen metabolism and confers aggressive prostate cancer in targeted mice and patients. Gene Dev 27, 683-698 (2013).
38. Gasi, D. et al. Overexpression of full-length ETV1 transcripts in clinical prostate cancer due to gene translocation. Plos One 6, e16332 (2011).
39. Kragesteen, B. K. et al. Dynamic 3D chromatin architecture contributes to enhancer specificity and limb morphogenesis. Nat Genet 50, 1463-1473 (2018).
40. Despang, A. et al. Functional dissection of the Sox9-Kcnj2 locus identifies nonessential and instructive roles of TAD architecture. Nat Genet 51, 1263-1271 (2019).
41. Kraft, K. et al. Serial genomic inversions induce tissue-specific architectural stripes, gene misexpression and congenital malformations. Nat Cell Biol 21, 305-310 (2019).
42. Zheng, M. et al. Multiplex chromatin interactions with single-molecule precision. Nature 566, 558-562 (2019).
43. Quinodoz, S. A. et al. Higher-Order Inter-chromosomal Hubs Shape 3D Genome Organization in the Nucleus. Cell 174, 744-757 e724 (2018).
44. Ay, F., Bailey, T. L. & Noble, W. S. Statistical confidence estimation for Hi-C data reveals regulatory chromatin contacts. Genome Res 24, 999-1011 (2014).
45. Wang, Y. et al. The 3D Genome Browser: a web-based browser for visualizing 3D genome organization and long-range chromatin interactions. Genome Biol 19, 151 (2018).
46. Boeva, V. et al. Control-FREEC: a tool for assessing copy number and allelic content using next-generation sequencing data. Bioinformatics 28, 423-425 (2012).
47. Liu, J. et al. An Integrated TCGA Pan-Cancer Clinical Data Resource to Drive High-Quality Survival Outcome Analytics. Cell 173, 400-416 e411 (2018).
48. Wang, X. T., Cui, W. & Peng, C. HiTAD: detecting the structural and functional hierarchies of topologically associating domains from chromatin interactions. Nucleic Acids Res 45, e163 (2017).
49. Abdennur, N. & Mirny, L. A. Cooler: scalable storage for Hi-C data and other genomically labeled arrays. Bioinformatics 36, 311-316 (2020).
50. Xu, W. et al. CoolBox: a interactive genomic data explorer for Jupyter Notebook. bioRxiv, 614222 (2019).
51. Ramirez, F. et al. deepTools2: a next generation web server for deep-sequencing data analysis. Nucleic Acids Res 44, W160-165 (2016).
52. Kaul, A., Bhattacharyya, S. & Ay, F. Identifying statistically significant chromatin contacts from Hi-C data with FitHiC2. Nat Protoc 15, 991-1012 (2020).

1. A method for predicting SV-induced chromatin interactions, which can be used to identify critical oncogenic regulatory elements for tumorigenesis and potentially reveal therapeutic targets, said method comprising the steps of:
inferring copy number from Hi-C map;
balancing separate matrix and correcting CNV effect;
simulating CNV effects on a normal Hi-C map;
detecting rearranged fragment, filtering SV, and assembling complex SV;
normalizing allele;
performing machine-learning based loop detection;
identifying Neo-TAD;
iisualizing reconstructed Hi-C map and genome browser tracks.

2. The method of claim 1, wherein a two-step process is used to detect CNV directly from a Hi-C map at high resolution, said process comprising:
a GAM being utilized to model the non-linear relationship between the one-dimensional coverage profile and different biases commonly observed in a Hi-C experiment;
an HMM based segmentation algorithm being utilized to determine the boundaries of CNV segments from the initial CNV profile.

3. The method of claim 2, wherein the HMM is built using the pomegranate Python package with each state approximated by a 2-component Gaussian mixture.

4. The method of claim 3, wherein the Baum-Welch algorithm is used to estimate the parameters of transition and emission, and the Viterbi algorithm is used to predict the state (copy number) of each bin.

5. The method of claim 4, wherein the consecutive bins assigned with the same state are merged together to form a CNV segment.

6. The method of claim 1, wherein a modified ICE procedure is used by applying ICE to regions with different copy numbers separately.

7. The method of claim 1, wherein a mathematical framework is utilized to simulate the effect of abnormal karyotypes on a diploid Hi-C dataset.

8. The method of claim 1, wherein the stretch of the rearranged fragments is recognized by locating the corner square block on the correlation matrix.

9. The method of claim 8, wherein the principal component analysis is performed, and the boundary loci are identified where the sign of the first eigenvector/principal component changes.

10. The method of claim 1, wherein the SVs are filtered by a linear regression model fitted between the global average contact frequencies and the local distance averaged contact frequencies for each SV.

11. The method of claim 1, wherein the complex SVs are assembled after being detected by checking the overlap of rearranged fragments between simple SVs.

12. The method of claim 11, wherein a directed graph is built with each node representing a simple SV, and each edge representing an overlap of rearranged fragments in consistent orientations.

13. The method of claim 12, wherein the shortest paths between any two nodes of the graph are calculated by the Dijkstra's algorithm and each such path is defined as a candidate complex SV.

14. The method of claim 13, wherein the linear regression-based method is applied to determine the assembly continuity.

15. The method of claim 14, wherein candidates are removed if the whole path or part of the path form a circular assembly are removed, or if they are redundant.

16. The method of claim 1, wherein a linear regression-based model is utilized to minimize the differences between local distance decay curves and the whole-genome distance decay curve for allele normalization.

17. The method of claim 1, wherein a machine-learning based framework referred to as Peakachu is applied for loop detection.

18. The method of claim 17, wherein the higher probability score computed by the CTCF or the H3K27ac model was recorded for each pixel of the reconstructed Hi-C map.

19. The method of claim 18, wherein the same pooling algorithm used in Peakachu was applied to each SV region independently to select the best-scored loop contacts from each cluster after filtering with a pre-defined probability threshold.

20. The method of claim 1, wherein neo-TAD detection algorithm is based on the directionality index (DI) and takes two steps comprising:
calculating DI along the reference genome (hg38) and used as input to learn a global HMM model;
recalculating DI on the allele normalized Hi-C map of each local assembly and use the model trained above and the Viterbi algorithm to predict the state of each bin.


SEQUENCE LISTING (16 sequences)

SEQ ID NO: 1 - DNA, 86 bp, Homo sapiens
caagcagaagacggcatacgagagatgtagccgtgactggagttcagacgtgtgctcttccgatctgttgcctgctctctgccagt

SEQ ID NO: 2 - DNA, 78 bp, Homo sapiens
aatgatacggcgaccaccgagatctacactctttccctacacgacgctcttccgatcttccactctccctgggactct

SEQ ID NO: 3 - RNA, 16 nt, Homo sapiens
gacgaggagacagacc

SEQ ID NO: 4 - RNA, 15 nt, Homo sapiens
gggaggaagacacac

SEQ ID NO: 5 - RNA, 10 nt, Homo sapiens
gaagcacacc

SEQ ID NO: 6 - RNA, 12 nt, Homo sapiens
aggagaggaaag

SEQ ID NO: 7 - RNA, 13 nt, Homo sapiens
agacccaaagccc

SEQ ID NO: 8 - RNA, 13 nt, Homo sapiens
cagaggccaaaac

SEQ ID NO: 9 - RNA, 15 nt, Homo sapiens
aagccaacagaccag

SEQ ID NO: 10 - RNA, 13 nt, Homo sapiens
caacaaaacaccg

SEQ ID NO: 11 - DNA, 22 bp, Homo sapiens
tttccttctttttaatttagcc

SEQ ID NO: 12 - DNA, 20 bp, Homo sapiens
aaaaaccctcaaaaccaaaa

SEQ ID NO: 13 - DNA, 23 bp, Homo sapiens
tcattaattatctcagtgtccat

SEQ ID NO: 14 - DNA, 22 bp, Homo sapiens
cactcagaaaagtaaacaccaa

SEQ ID NO: 15 - DNA, 24 bp, Homo sapiens
actccatttatatgaggcaagaag

SEQ ID NO: 16 - DNA, 22 bp, Homo sapiens
ttgtttgatgtctccatcgaat